(19) United States
(12) Plant Patent Application Publication
(10) Pub. No.: US 2015/0033427 P1
(43) Pub. Date: Jan. 29, 2015

(54) CALIBRACHOA PLANT NAMED 'KLECA13249'

(50) ;

(71) Applicant: Klemm+Sohn GmbH & Co. KG, Stuttgart (DE)

(72) Inventors: Nils Klemm, Stuttgart (DE); Anita Stöver, Ostfildern (DE)

(73) Assignee: Klemm+Sohn GmbH & Co. KG, Stuttgart (DE)

(21) Appl. No.: 13/987,415

(22) Filed: Jul. 23, 2013

(51) Int. Cl. A01H 5/00

(52) U.S. Cl. PLT/413

(57) ABSTRACT

A new Calibrachoa plant particularly distinguished by having pink flowers with purple veins and a semi-trailing plant habit, is disclosed.

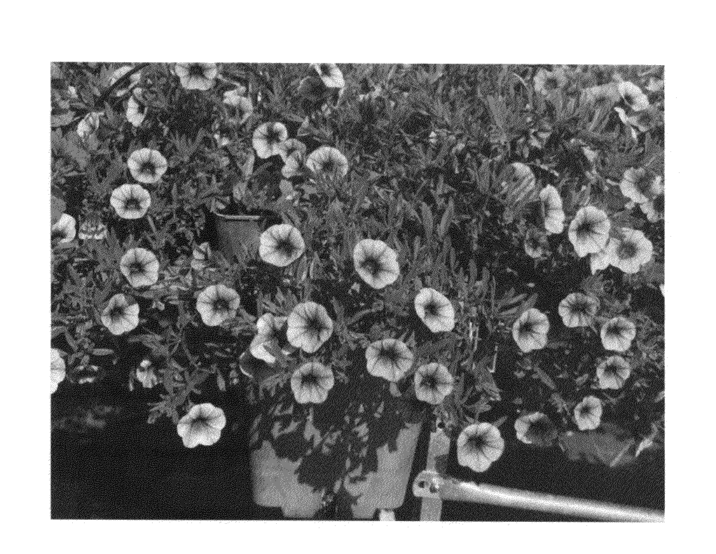

### GENUS AND SPECIES

Calibrachoa sp.

### VARIETY DENOMINATION

‘KLECA13249’

### BACKGROUND OF THE NEW PLANT

The present invention comprises a new and distinct variety of Calibrachoa, botanically known as Calibrachoa sp., and hereinafter referred to by the variety name ‘KLECA13249’. ‘KLECA13249’ originated from a controlled cross between the proprietary female Calibrachoa variety ‘CA 08 0402’ (unpatented) and the proprietary male Calibrachoa variety ‘CA 08 0252’ (unpatented) in May 2009.

In May 2009, seed was collected from the controlled cross between the two Calibrachoa parental lines, ‘CA 08 0402’ and ‘CA 08 0252’ in Stuttgart, Germany. The seeds were sown and plants were grown for evaluation. A single plant line was selected in May 2010 which had light pink flowers with purple veins and was subsequently named ‘KLECA13249’. In May 2010, ‘KLECA13249’ was first vegetatively propagated by tip cuttings. ‘KLECA13249’ was found to reproduce true to type in successive generations of asexual propagation via tip cuttings.

### CROSS REFERENCE TO RELATED APPLICATIONS

Plant Breeder's Rights for ‘KLECA13249’ were applied for in Canada on Jan. 23, 2013, Application No. 13-7853. ‘KLECA13249’ has not been made publicly available or sold more than one year prior to the filing date of this application.

### SUMMARY

The following are the most outstanding and distinguishing characteristics of this new variety when grown under normal horticultural practices in Stuttgart, Germany.

1. Light pink flowers with purple veins; and

2. A semi-trailing plant habit.

### DESCRIPTION OF THE PHOTOGRAPH

This new Calibrachoa plant is illustrated by the accompanying photograph which shows the foliage, buds, and a close-up of the flowers. The photograph is of a five-month-old plant grown in Stuttgart, Germany in May 2012. The colors shown are as true as can be reasonably obtained by conventional photographic procedures.

### DESCRIPTION OF THE NEW VARIETY

The following detailed descriptions set forth the distinctive characteristics of ‘KLECA13249’. The data which define these characteristics were collected from asexual reproductions carried out in Stuttgart, Germany. Data was collected on 5-month-old plants grown in a controlled greenhouse environment in Stuttgart, Germany in May 2013. Color references are to The R.H.S. Colour Chart of The Royal Horticultural Society of London (R.H.S.) (2007).
Classification:
   Family.—Solanaceae.
   Botanical.—Calibrachoa sp.
   Common.—Calibrachoa.
   Designation.—‘KLECA13249’.
Parentage:
   Female parent.—Proprietary Calibrachoa line ‘CA 08 0402’ (unpatented).
   Male parent.—Proprietary Calibrachoa line ‘CA 08 0252’ (unpatented).
Plant:
   Life cycle.—Annual.
   Form and habit.—Semi-trailing.
   Height (from soil line to top of foliage).—5.0 cm.
   Spread.—33.0 cm.
   Propagation.—Vegetatively propagated by tip cuttings.
   Time to produce a rooted cutting.—3 weeks.
Stems:
   Color.—RHS 144D and RHS 200A.
   Pubescence.—Little.
   Pubescence color.—RHS NN155B.
   Diameter.—7.0 cm.
   Length from soil line to first node.—1.9 cm.
   Total length.—18.0 cm (measured from top of the stem to the base of the apical branch).
   Internode length.—2.1 cm.
Leaves:
   Arrangement.—Alternate, simple, and sessile.
   Shape.—Elliptic.
   Apex.—Obtuse to slightly acute.
   Base.—Attentuate.
   Margin.—Entire.
   Surface.—Smooth.
   Surface pubescence.—Absent.
   Venation.—Pinnate.
   Venation color.—Upper surface: RHS 144A Lower surface: RHS 144C.
   Length.—2.4 cm.
   Width.—0.6 cm.
   Color.—Upper surface: RHS 144A and RHS 187A Lower surface: RHS N187A.
   Fragrance.—Absent.
Flowers:
   Flowering habit.—Flower attitude is semi-erect, single, axillary.
   Flower type.—Determinate inflorescence.
   Flowering requirements.—A minimum of light sun is required.
   Duration of flowers.—4 days.
   Corolla.—Smooth texture.
   Shape.—Funnel-shaped.
   Fragrance.—Absent.
Flower buds:
   Surface.—Smooth.
   Length.—1.9 cm.
   Diameter.—0.6 cm.
   Shape.—Irregular to oblong.
   Color.—RHS N92A.
Peduncle:
   Length.—1.7 cm.
   Diameter.—0.1 cm.
   Color.—RHS 144B.
   Texture.—Smooth.
   Anthocyanin color.—RHS 200A.
Flower description:
   Flower depth.—3.3 cm.
   Flower tube length.—1.6 cm.
   Flower tube diameter.—0.8 cm.
   Flower diameter.—4.5 cm.
   Pedicel color.—RHS 200A.
   Calyx.—Funnel-shaped.
Sepals:
   Shape.—Lanceolate.
   Apex.—Acute.
   Margin.—Entire.
   Length.—1.5 cm.
   Diameter.—0.3 cm.
   Sepal color.—Upper surface: RHS 144B Lower surface: RHS 200C.
Petals:
   Shape.—Heart-shaped.
   Length.—2.0 cm.
   Apex.—Obverse.
   Margin.—Entire, weak undulation.
   Base.—Open and fused into the tube.
   Pubescence.—Absent.
   Lobe length.—1.6 cm.
   Lobe width.—2.0 cm.
   Lobe color.—Upper surface: RHS 69A and RHS 70B Lower surface: RHS 70B and RHS N92A.
   Corolla tube color.—Inner: RHS 162B Outer: RHS N92A.
   Fragrance.—Absent.
Reproductive organs:
   Stamen number.—5.
   Stamen color.—Anther color: RHS 13B Filament color: RHS 1C Pollen color: RHS 13B.
   Ovary.—Placenta arrangement: Ovate Pistil number: 1 Pistil length: 0.1 cm Stigma color: RHS 154A Style length: 0.8 cm Style color: RHS 149B.
Fruit and seed set: Little seed production.
Disease and pest/insect resistance: None observed.

### COMPARISON WITH PARENTAL LINES AND KNOWN VARIETY

‘KLECA13249’ is distinguished from the female and male parental lines as described in Table 1.

TABLE 1
Comparison with Parental Line

| | | Female parent | Male Parent |
|---|---|---|---|
| Trait | ‘KLECA13249’ | ‘CA 08 0402’ | ‘CA 08 0252’ |
| Flower | RHS 69N and RHS | Single color, orange | Single color, red |
| color | 70B | | |

‘KLECA13249’ is most similar to the Calibrachoa plant named ‘KLECA10216’ (U.S. Plant Pat. No. 22,893). Differences between the two varieties are described in Table 2.

TABLE 2
Comparison with Similar Variety

| Trait | ‘KLECA13249’ | ‘KLECA10216’ |
|---|---|---|
| Flower Color | RHS 69N and RHS 70B | RHS 75C and RHS 61B |

We claim:

1. A new and distinct variety of Calibrachoa plant named ‘KLECA13249’ as shown and described herein.
